(19) United States
(12) Patent Application Publication
(10) Pub. No.: US 2022/0096655 A1
(43) Pub. Date: Mar. 31, 2022

(54) PLASMA POLYMER NANOPARTICLES CARRYING AGENTS

(71) Applicant: THE HEART RESEARCH INSTITUTE LTD, NEWTOWN, NEW SOUTH WALES (AU)

(72) Inventors: Steven Garry WISE, Newtown, New South Wales (AU); Miguel CORREIA DOS SANTOS, Newtown, New South Wales (AU); Richard Philip TAN, Newtown, New South Wales (AU); Jui Chien HUNG, Newtown, New South Wales (AU)

(21) Appl. No.: 17/429,754

(22) PCT Filed: Feb. 11, 2020
(86) PCT No.: PCT/AU2020/050111

§ 371 Date: Aug. 10, 2021

(30) Foreign Application Priority Data
Feb. 11, 2019 (AU) 2019900427

(51) Int. Cl. A61K 47/69; A61K 31/192; A61K 31/337; A61K 38/20; A61K 31/351; A61K 47/64

(52) U.S. Cl. CPC A61K 47/6933; A61K 31/192; A61K 31/337; B82Y 5/00; A61K 38/2066; A61K 31/351; A61K 47/642; A61K 38/2026

(57) ABSTRACT

The present disclosure relates to the field of nanoparticles, conjugates thereof and their use in methods of treatment or prevention of vascular inflammation. The present disclosure also relates to methods of delivering an agent to a region of a blood vessel in a patient, comprising: a) conjugating the agent to a nanoparticle to produce a conjugate; and b) delivering the conjugate to the region of the blood vessel.

### CROSS-REFERENCE TO RELATED APPLICATIONS

The present application claims priority from Australian Provisional Patent Application No 2019900427 filed on 11 Feb. 2019, the content of which is incorporated herein by reference.

### TECHNICAL FIELD

The present disclosure relates to the field of nanoparticles, conjugates thereof and their use in methods of treatment or prevention of vascular inflammation.

### BACKGROUND

Coronary artery atherosclerosis disease is the leading cause of death and disability in Western society. Occlusion of coronary vessels results in reduced blood flow to heart muscle, damage to this tissue and ultimately myocardial infarction. However, the long-term performance of medical devices used in the treatment of atherosclerosis, such as drug eluting stents and angioplasty balloons, is limited by chronic inflammation at the injury site.

The long-term success of surgical and vascular interventions is limited by neointimal hyperplasia (NIH). In an artery, NIH is the thickening of the arterial intimal layer after an injury such as angioplasty, stenting or surgical repair. NIH is also used to describe the thickening of venous and prosthetic bypass grafts that leads to reduced lumen diameter and flow and, ultimately, graft occlusion and thrombosis. NIH affects all forms of vascular grafts, including both venous and prosthetic conduits used in coronary and peripheral arterial bypass, and arteriovenous fistulae (AVF) created for hemodialysis access.

Restenosis is a common adverse event of endovascular procedures such as insertion of a stent, balloon angioplasty, or vascular surgery. One of the causative factors of restenosis is an inflammatory immune response triggered in response to the endovascular procedure. It is the recurrence of stenosis, which is the narrowing of a blood vessel, leading to reduced blood flow.

Thus, there remains a need for methods of delivering biologically active agents and drugs, such as anti-inflammatory agents, to blood vessels.

### SUMMARY

The present disclosure describes new therapies for localizing a biologically active agent or drug in a region of a blood vessel, for example for regulating inflammation or promoting healing or treating or preventing disease in blood vessels. In particular, the inventors have identified that nanoparticles or nanoP³ can bind agents such as biologically active agents, drugs and imaging agents, whilst retaining their bioactivity both in vitro and in vivo.

Accordingly, in one aspect, the present disclosure provides a method of delivering an agent to a region of a blood vessel in a patient, comprising:
a) conjugating the agent to a nanoparticle to produce a conjugate; and
b) delivering the conjugate to the region of the blood vessel.

In another aspect, the present disclosure provides a method of regulating inflammation or promoting healing in a region of a blood vessel in a patient, comprising:
a) conjugating a biologically active agent to a nanoparticle to produce a conjugate; and
b) delivering the conjugate to the region of the blood vessel.

In another aspect, the present disclosure provides a method of retaining a biologically active agent to a region in a blood vessel in a patient for a period of at least 14 days comprising:
a) conjugating the biologically active agent to a nanoparticle to produce a conjugate;
and b) delivering the conjugate to the blood vessel.

In another aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 5 nm to about 500 nm;
and a biologically active agent selected from the group consisting of: an anti-inflammatory cytokine; anti-inflammatory drug; a statin drug and an anti-proliferative drug.

In another aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm;
and interleukin-10.

In another aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm;
and Sirolimus.

In another aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm;
and Sulindac.

In another aspect, the present disclosure provides a method of treating or preventing vascular injury or vascular disease comprising delivering the conjugate disclosed herein to a region of a blood vessel in a patient in need thereof.

In another aspect, the present disclosure provides the use of the conjugate disclosed herein in the manufacture of a medicament for treating or preventing vascular injury or vascular disease in a patient in need thereof.

### BRIEF DESCRIPTION OF THE DRAWINGS

The following figures form part of the present specification and are included to further demonstrate certain aspects of the present disclosure. The disclosure may be better understood by reference to one or more of these figures in combination with the detailed description of specific embodiments presented herein.

FIG. 1 shows the evaluation of NP3 conjugated IL-4 (NP3+IL-4) as a therapeutic treatment to mitigate cardiovascular disease. The delivery of therapeutic cargo directly onto sites of decreased vasculature was investigated. A) Rapid conjugation of therapeutic compounds and increased bioavailability of these compounds when delivered in vivo is possible through the intrinsic properties of the NP3 platform. B) M1 macrophages which drive the pathologies of vessel injury/cardiovascular disease are pro-inflammatory. Directing the phenotype of these macrophages to their M2 anti-inflammatory state has the potential to mitigate the further progression of vessel injury and promote disease regression. Various cytokines from the interleukin-family facilitate this shift in phenotype from M1 to M2, including IL-4 and IL-10. C) Using a rat carotid model of vessel the efficacy of NP3 bound interleukins to treat cardiovascular pathology was determined.

FIG. 2 shows the loading capacity of IL-4 (blue) and IL-10 (red) on NP3. IL-4 and IL-10 were tagged using a Cy5 label and conjugated onto NP3. Washes were quantified for fluorescence readings of the Cy5 label to determine how much cargo was left in solution (unconjugated to NP3) to determine NP3 loading. A) Total cargo bound as a function of cargo in solution. B) Binding efficiency of IL-4 and IL-10 onto NP3 as a function of loading capacity. C) Emission spectrum of bound IL-4 and IL-10 to confirm binding of IL-4 and IL-10.

FIG. 3 shows validation of NP3+IL-4 on M2 macrophage polarisation in vitro. A) Scanning electron microscopy (SEM) of RAW 264.76 mouse macrophages show that, compared to untreated controls and NP3 only, NP3+IL-4 treatment increases spreading (top row) and surface roughness (bottom row), consistent with M2 activation. B) Using confocal microscopy, immunostaining of the highly expressed M2 enzyme, arginase-1 (ARG-1) (green), showed that NP3+IL-4 significantly upregulated the expression of ARG-1, further indicating robust M2 activation compared to control and NP3 only.

FIG. 4 shows an in vivo model of vessel injury and NP3 retention. A) Procedural workflow of vessel injury: 1. Microintraocular forceps are inserted into ligated/isolated carotid rat segments. 2. Forceps are expanded and rotated 360° to mimic balloon injury overexpansion injury/denudation. 3. Forceps are removed, and through the same incision a small gauge catheter is inserted. 4. NP3+IL-4 solutions are delivered through the catheter and are allowed to incubate in the isolated vessel for 2 minutes. 5. The incision is sutured, and blood flow is restored. B) Tracking of IL-4 retention in the isolated vessel using a Cy5 tag shows that free IL-4 is immediately washed away from the vessel wall following restoration of blood flow. However, when bound to NP3 (NP3+IL-4), IL-4 is significantly retained in the vessel and this persists at substantial levels after 5 days.

FIG. 5 shows the inhibitory mechanisms of neointima formation. A) Immunostaining of the presence of M2 macrophages (yellow/green) in treated carotid segments shows a significant increase in NP3+IL-10 groups compared to denuded, NP3+IL-4, and free IL-10. B) Evaluation of repair of the damaged endothelium by immunostaining shows that both NP3+IL4 and NP3+IL-10 restore full endothelium integrity by 14 days after injury, however this is absent when treated with free IL-10.

FIG. 6 shows the analysis of two-week neointima formation following therapeutic NP3 delivery. A) Representative histology photos showing the extent of neointima formation in each of the treatment groups. B) Quantification of neointima formation percentage in three segments along the length of the treated carotid segment. ‘Proximal’ and ‘Distal’ labels indicate orientation of the vessel anastomoses in proximity to the heart. Denuded injury results in roughly 60% vascular occlusion after two weeks. This is significantly reduced to approximately 35% and 20% in NP3+IL-4 and NP3+IL10 groups, respectively. Free IL-10 and NP3 alone have no significant effects on vascular occlusion, suggesting that the NP3 platform facilitates the therapeutic benefits of IL-10.

FIG. 7 shows in vivo analysis of neointimal hyperplasia in a rat carotid injury model using hematoxylin & eosin (H&E) staining. Compounds tested include the anti-inflammatory cytokine interleukin-10 (IL-10), the anti-proliferative drug Sirolimus, and the non-steroidal anti-inflammatory drug (Sulindac), delivered freely or conjugated to 200 nm NP3. When delivered on 200 nm NP3, all treatments show suppression of neointimal hyperplasia compared to their respective freely delivered controls.

FIG. 8 shows in vivo analysis of vessel re-endothelialisation in a rat carotid injury model using von Willebrand factor (vwf) staining. Compounds tested include the anti-inflammatory cytokine interleukin-10 (IL-10), the anti-proliferative drug Sirolimus, and the non-steroidal anti-inflammatory drug (Sulindac), delivered freely or conjugated to 200 nm NP3. NP3 delivered IL-10 stimulates vessel healing (endothelialisation). Surprisingly, NP3 delivered Sirolimus also stimulates vessel healing.

FIG. 9 shows in vivo analysis of vessel inflammation/macrophage polarisation in a rat carotid injury model using mannose receptor (CD206) co-staining with the CD68 cell surface receptor. Compounds analysed include the anti-inflammatory cytokine interleukin-10 (IL-10) and the non-steroidal anti-inflammatory drug (Sulindac), delivered freely or conjugated to 200 nm NP3. NP3-IL-10 stimulates anti-inflammatory responses by promoting M2 macrophage polarisation.

FIG. 10 shows the performance outcomes of IL-10 conjugated 200 nm NP3 in vivo in a rabbit model of iliac arterial injury. A) rabbit model of iliac arterial injury. B) H&E staining of neointimal hyperplasia. C) CD31 staining of thrombosis (white dotted line). D) CD68 staining of inflammatory macrophage infiltration (white staining).

FIG. 11 shows the performance outcomes of IL-10 conjugated 200 nm NP3 in vivo in a rabbit model of iliac arterial injury. A) H&E staining analysis shows that IL-10+NP3 treated vessels had reduced hyperplasia over seven days compared to untreated controls. B) CD31 staining analysis reveals that the incidence of thrombosis increases over seven days and this is significantly reduced in IL-10+NP3 treated vessels. C) CD68 staining of vessel inflammation shows that IL-10+NP3 reduces the infiltration of pro-inflammatory macrophages and reduces inflammation over seven days compared to untreated controls.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### DETAILED DESCRIPTION

### General Techniques and Definitions

Unless specifically defined otherwise, all technical and scientific terms used herein shall be taken to have the same meaning as commonly understood by one of ordinary skill in the art (e.g. immunology, molecular biology, immunohistochemistry, biochemistry, oncology, and pharmacology).

The present disclosure is performed without undue experimentation using, unless otherwise indicated, conventional techniques of molecular biology, recombinant DNA technology, immunology and pharmacology. Such procedures are described, for example in Sambrook, Fritsch & Maniatis, Molecular Cloning: A Laboratory Manual, Cold Spring Harbor Laboratories, New York, Fourth Edition (2012), whole of Vols I, II, and III; DNA Cloning: A Practical Approach, Vols. I and II (D. N. Glover, Second Edition., 1995), IRL Press, Oxford, whole of text; Oligonucleotide Synthesis: A Practical Approach (M. J. Gait, ed, 1984) IRL

Press, Oxford, whole of text, and particularly the papers therein by Gait, pp1-22; Atkinson et al, pp35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984) and Methods In Enzymology (S. Colowick and N. Kaplan, eds., Academic Press, Inc.), whole of series.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment or embodiment of the present disclosure may be applied mutatis mutandis to any other aspect or embodiment or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise. For example, a reference to “a bacterium” includes a plurality of such bacteria, and a reference to “an allergen” is a reference to one or more allergens.

Herein the term “about” encompasses a 10% tolerance in any value(s) connected to the term. For the avoidance of doubt, it is to be understood that the term “about” includes a specific reference to the integer (e.g. “about 10” is to be understood as including an explicit reference to 10).

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

The present inventors have demonstrated in WO2018/112543, the entire content of which is herein incorporated by reference, the production of nanoparticulate materials, described herein as “nanoP³”, “nanoP3” “NanoP³”, “nanoP³ material”, “NanoP³ material” or “NP3”.

These nanoP³ materials can act as a class of versatile and multifunctional nanocarriers which may be readily functionalised. The nanoP³ material can be conjugated to a large range of biomolecules and drugs through reaction with radicals embedded within the nanoP³ material which diffuse to the surface of the nanoP³ material and/or by reaction with moieties/functional groups formed on the surface of the nanoP³ material, or conjugates thereof.

The present inventors have discovered that delivery of biologically active agents and/or imaging agents conjugated to a nanoparticle may prolong the bioavailability and/or persistence of the agents and drugs delivered in vivo in a region of blood vessel, for example at an injury site in a blood vessel, as compared to the agents when freely injected into the region of the blood vessel (i.e. the agent is delivered in a form that is not conjugated to a nanoparticle as described herein).

Thus, in some embodiments, the conjugate is retained in the region of the blood vessel for a period greater than the period unconjugated biologically active agent would be retained in the region of the blood vessel.

The present disclosure provides methods for localizing biologically active agents and regulating inflammation in blood vessels in response to vascular intervention. The present disclosure also provides methods of treating or preventing vascular disease or injury.

### Inflammation, Restenosis and Neointimal Hyperplasia

The methods of the present disclosure may be used to deliver and/or localize agents, for example biologically active agents or imaging agents to a region of a blood vessel in a patient. Alternatively or in addition, the methods of the disclosure may be used to regulate inflammation or promote healing in a region of a blood vessel in a patient.

In one aspect, the present disclosure provides a method of delivering an agent to a region of a blood vessel in a patient, comprising:
a) conjugating the agent to a nanoparticle to produce a conjugate; and
b) delivering the conjugate to the region of the blood vessel.

In one embodiment, the nanoparticle is a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 5 nm to about 500 nm;

In one embodiment, the agent is a biologically active agent.

In another embodiment, the agent is an imaging agent.

In one aspect, the present disclosure provides a method of regulating inflammation in a region of a blood vessel in a patient, comprising:
a) conjugating a biologically active agent to a nanoparticle to produce a conjugate; and
b) delivering the conjugate to the region of the blood vessel.

In one example, the regulation of inflammation is through macrophage polarization.

In one aspect, the present disclosure provides a method of promoting healing in a region of a blood vessel in a patient, comprising:
a) conjugating a biologically active agent to a nanoparticle to produce a conjugate; and b) delivering the conjugate to the region of the blood vessel.

Inflammation is triggered when tissues are exposed to one or more of a variety of insults. This response consists of a cascade of events that includes the release of various chemical mediators and recruitment of circulating blood cells (platelets and leukocytes) to the site of injury and their subsequent activation.

Without wishing to be bound by theory, restenosis is believed to be a natural healing process in response to the arterial injury that occurs during all types of angioplasty procedures. This very complex healing process results in intimal hyperplasia, more specifically migration and proliferation of medial smooth muscle cells (SMC). The problem associated with this arterial healing process is that in some instances, it does not shut off. The artery continues to “heal” until it becomes occluded. It should be noted that restenosis is not a re-deposition of the plaque-like cholesterol material that originally occluded the artery.

Without wishing to be bound by theory, it is believed that successful angioplasty of stenotic lesions produces cracking of the plaque, dissection into the media, denudation and destruction of endothelial cells, exposure of thrombogenic collagen, released tissue thromboplastin, and an increased loss of prostacyclin production, leading to the aggregation of active platelets.

Activated platelets release several mitogens including platelet derived growth factor (PDGF), epidermal growth factor, and transforming growth factor. PDGF has both mitogenic and chemotactic properties and thus, may induce both mitigation of SMC from the medial layer to the intimal layer as well as proliferation (Intimal hyperplasia). PDGF causes SMC proliferation by binding to specific PDGF receptors. Once the PDGF is bound to the receptors, deoxyribose nucleic acid (DNA) synthesis occurs and new cells are replicated.

Minor endothelial injury may cause platelet adhesion and activation with the resultant release of PDGF. Thus, even the deposition of a monolayer of platelets may be sufficient to induce SMC proliferation.

Deeper arterial injury which is sometimes associated with complex stenotic lesions leads to more extensive platelet deposition and activation which may cause an even greater availability of the mitogenic factors. Thus, increased SMC proliferation and intimal hyperplasia. Arterial injury from angioplasty may result in release of PDGF-like compounds from not only platelets but also macrophages, monocytes, endothelial cells, or SMC's themselves.

Activated SMC from human atheroma or following experimental arterial injury secrete PDGF-like molecules which appears to lead to the self-perpetuation of SMC proliferation by the release of their own PDGF-like substances. Thus, any or all of the cells which can secrete PDGF relate substances (platelets, macrophages, monocytes, endothelia, and smooth muscle cells) may contribute to the cascading effect of restenosis after angioplasty.

One way of preventing restenosis is to stop the proliferation of smooth muscle cells. Thus, without wishing to be bound by theory, some ways to stop restenosis may be to:
Reduce the adhesion and aggregation of the platelets at the arterial injury site;
Block the expression of growth factors and their receptors;
Develop competitive antagonists of the above growth factors;
Interfere with the receptor signalling in the responsive cell; or
Inhibit smooth muscle proliferation.

### Nanoparticle

The term “nanoparticle” may be used interchangeably with “nanoP³” or “NP3”. The term “nanoparticle” or “nanoP³” or “NP3” refers to a nanoparticulate material having a size less than 100 micron unless otherwise specified or clear from the context in which it is used. For example, the nanoP³ may have a size of between about 50 to 500, 100 to 500, 200 to 500, 5 to 200, 5 to 100, 5 to 50, 5 to 20, 20 to 100, 100 to 300 or 200 to 400 nm, e.g., about 5, 10, 20, 30, 40, 50, 100, 150, 200, 250, 300, 350, 400, 450 or 500 nm, or in a range of about 5 to about 400 nm, or about 5 to about 300 nm, or about 5 to about 200 nm, or about 5 to about 100 nm, or about 50 to about 100 nm, or about 100 to about 500 nm, or about 150 to about 500 nm, or about 180 nm to about 500 nm, or about 100 to about 400 nm, or about 150 to about 400 nm, or about 180 to about 400 nm, or about 100 to about 300 nm, or about 150 to 300 nm, or about 180 to 300 nm, or about 100 to about 200 nm, or about 150 to about 200 nm, or about 180 to about 200 nm, or about 150 to about 250 nm, or about 180 to about 250 nm, or about 200 to about 400 nm, or about 200 nm to about 300 nm, or a mixture thereof. In one embodiment, the nanoP3 is about 200 nm. In another embodiment, the nanoP3 is between about 50 to about 100 nm. It will be appreciated that the sizes described herein refers to diameters or average diameters of the nanoparticulate material. The term “nanoP³” or “NP3” encompasses both “nanoparticulate polymers” and “aggregates” as defined herein unless otherwise specified or clear from the context in which it is used. Thus, for example, the sizes described above apply equally to the nanoparticles or aggregates of nanoparticles. In one preferred embodiment the nanoparticulate material comprises a plasma polymer. The plasma polymer may be formed by the condensation of fragments in a plasma, said material being capable of covalently coupling one or more compounds, for example one or more agents, including organic or organometallic species.

The nanoP3 material may be a homopolymer or a copolymer. Examples of suitable nanoP3 materials and methods for deriving suitable nanoP3 materials are described at page 21, line 2 to page 28, line 12 of PCT Publication no. WO2018/112543, which is herein incorporated by reference.

Nanoparticulate polymers may be formed in the presence of a gas from group 15, 16 or 17 of the periodic table, such as nitrogen. Fragments of this gas may be imported into the nanoparticulate polymer. For example, the presence of nitrogen may result in the presence of amine, imine or nitrile groups, or a mixture thereof in a nanoparticulate polymer or nanoP³ material. Thus, the nanoparticulate polymer disclosed herein may comprise nitrogen.

Nitrogen has been found to be suitable not only as a carrier but also as a reactive non-polymerisable gas. This means that nitrogen may also be incorporated in the nanoparticulate material, imparting particular physical-chemical properties to the resulting functionalised nanoparticulate material. Furthermore, nitrogen is also thought to enable different modes of nanoparticle formation that otherwise would not be possible if nitrogen was not used. It is expected that the inclusion of other gases, such as those in the same group of nitrogen, will also provide an extra degree of freedom in modulating nanoparticle formation mechanisms and physical-chemical properties.

In one embodiment the nanoP³ material is derived from a plasma comprising at least one monomer as described herein. Optionally the nanoP³ material is formed in the presence of a gas, for example nitrogen, wherein fragments of the gas are incorporated into the nanoparticulate polymer.

The nanoP³ material may have a nitrogen:carbon elemental ratio of about 0.01:1 to about 2:3. For example the nanoparticulate polymer may have a nitrogen:carbon elemental ratio of about 0.05 to about 1, or about 0.1 to about 1, or about 0.15 to about 1, or about 0.2 to about 1, or about 0.25 to about 1, or about 0.3 to about 1, or about 0.35 to about 1, or about 0.4 to about 1, or about 0.45 to about 1, or about 0.5 to about 1, or about 0.55 to about 1, or about 0.6 to about 1, or about 0.65 to about 1. Alternatively, the nanoparticulate polymer may have a nitrogen:carbon elemental ratio of about 0.1 to about 1:2. In one example, the nanoparticulate polymer may have a nitrogen:carbon elemental ratio of about 0.35 to about 0.5 or about 0.35 to about 1. In another example, the nanoparticulate polymer may have a nitrogen:carbon elemental ratio of about 0.38.

The nanoP³ materials described herein preferably comprise at least one binding site capable of binding one or more compounds, for example an organic or an organometallic compound, or a second species as defined herein.

In one embodiment the nanoP³ material described herein comprises at least binding site capable of binding one or more compounds, wherein the binding site comprises unpaired electrons which are capable of binding an organic or an organometallic compound, or a second species as defined herein.

The nanoP³ material may comprise unpaired electrons in the polymer. These unpaired electrons may be on or near the surface of the nanoparticles. The unpaired electrons may be at a depth of 40 nm or less within particles of the nanoparticulate material, or within about 30, 20 or 10 nm of the surface, or may be between about 10 and about 40 nm from the surface or between about 10 and 30, 20 and 40 or 20 and 30 nm from the surface, or about 1, 2, 3, 4, 5, 10, 15, 20, 25, 30, 35 or 40 nm from the surface. They may be at a variety of depths from about 0 to about 40 nm. In some instances they may be at depths of greater than 40 nm. They may be throughout the volume of the nanoparticulate material. This may render the material capable of reacting with a second species, such as an organic or organometallic species, so as to covalently couple said species to the nanoparticulate polymer and form a conjugate.

In an embodiment there is provided a nanoP³ material, particles thereof having a mean diameter of about 5 nm to about 500 nm, said nanoP³ material comprising an organic plasma polymer and said nanoP³ (nanoparticulate polymer or aggregate thereof) comprising unpaired electrons, thereby being capable of covalently couple with an organic or organometallic species.

In another embodiment the nanoparticulate material or nanoP³ material comprises at least one functional moiety which is capable of chemically or physically coupling a second species.

The size of the nanoP³ material may be measured by scanning electron microscopy, transmission electron microscopy, low angle laser light scattering, photon correlation spectroscopy, differential mobility analysis, or some other suitable technique. The particles of the nanoP³ material may have a narrow size distribution or a broad size distribution. The standard deviation of the particle size distribution may be between about 1% and about 500% of the mean particle size, or between about 1 and 200, 1 and 100, 1 and 50, 1 and 20, 1 and 10, 1 and 5, 1 and 2, 10 and 500, 20 and 500, 50 and 500, 100 and 500, 200 and 500, 10 and 100, 10 and 50 or 50 and 100%, e.g., about 1, 2, 3, 4, 5, 10, 20, 30, 40, 50, 60, 70, 80, 90, 100, 150, 200, 250, 300, 350, 400, 450 or 500%. In some instances they may be approximately monodispersed, i.e., all particles may be approximately the same size (e.g., within about 10%, or about 5%, or about 2% of the same diameter).

The nanoP³ material may comprise an organic plasma polymer. Plasma polymers are characterised by a heterogeneous, dense, highly crosslinked network. They may be amorphous. This plasma polymer may be generated by the reaction (e.g., ionisation and fragmentation), of active species generated in the plasma from the organic gas and other reactive gases in a gas mixture or by reactive species in the plasma/gas mixture resulting from the ionization and fragmentation of gases in the gas mixture.

The nanoP³ materials may be characterised via a number of methods including, but not limited to: electron paramagnetic resonance (EPR) spectroscopy, infrared spectroscopy (such as Fourier transform infrared spectroscopy), Raman spectroscopy, UV-VIS spectroscopy, elemental analysis (e.g., X-ray photoelectron spectroscopy), soft X-ray spectroscopy, determination of a zeta potential, nuclear magnetic resonance (NMR) spectroscopy, mass spectrometry, gel permeation chromatography, scanning electron microscopy (SEM), transmission electron microscopy (TEM), low angle laser light scattering, photon correlation spectroscopy, differential mobility analysis, elastic recoil detection analysis (ERDA), or neutron scattering.

The nanoP³ material (for example the nanoparticulate polymer or aggregates), may be characterised by one or more of the following features:
a broad electron paramagnetic resonance peak centred in a range of about 3470 G to about 3520 G, and/or corresponding to a g-factor in a range of about 2.001 to about 2.005;
a spin density measured by electron paramagnetic resonance within about 0 hours to about 2 hours post-synthesis in the range of about 10¹⁹ to about 10¹⁵ spins/cm³.
a spin density in the range of about 10¹⁷ to about 10¹⁵ spins/cm³ measured by electron paramagnetic resonance within about 0 hours to about 240 hours post synthesis.
one or more absorbance bands in an infrared spectrum centred:
   in a range of about 3680- about 2700 cm⁻¹;
   in a range of about 1800- about 1200 cm⁻¹;
   in a range of about 2330- about 2020 cm⁻¹;
   in a range of about 1200- about 1010 cm⁻¹; and/or
   in a range of about 1010- about 700 cm⁻¹;
one or more absorbance bands in an infrared spectrum centred:
   in a range of about 3600-3100 cm⁻¹; and/or
   in a range of about 3100-2700 cm⁻¹;
a zeta potential in a range of from about −100 mV to about +100 mV;
a zeta potential in a range of from about −80 mV to about +80 mV measured in a solution within a pH range of about 2 to about 10; or
a nitrogen:carbon elemental ratio of about 0.1:1 to about 2:3.

In one embodiment, the nanoP³ material, nanoparticulate polymer or aggregate is characterised using EPR spectroscopy. The nanoparticulate polymer or aggregate may show a broad electron paramagnetic resonance peak centred in a range of about 3470 G to about 3520 G, and/or corresponding to a g-factor in a range of about 2.001 to about 2.005.

In one embodiment the nanoP³ material, nanoparticulate polymer or aggregate has a spin density measured by EPR spectroscopy within about 0 hours to about 2 hours post-synthesis in the range of about 10¹⁹ to about 10¹⁵ spins/cm³. The measurement may be made: about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, or about 120 minutes, post-synthesis.

In one embodiment the nanoP³ material, nanoparticulate polymer or aggregate has a spin density in the range of about 10¹⁷ to about 10¹⁵ spins/cm³ measured by electron paramagnetic resonance within about 0 hours to about 240 hours post-synthesis. The measurement may be made: about 0.5, about 1, about 2, about 4, about 5, about 6, about 8, about 10, about 20, about 30, about 40, about 50, about 60, about 70, about 80, about 90, about 100, about 110, about 120, about 130, about 140, about 150 hours, about 160, about 170, about 180, about 190, about 200, about 210, about 220, about 230, or about 240 hours, post-synthesis.

In another embodiment, the nanoP³ material, nanoparticulate polymer or aggregate is characterised using infrared spectroscopy (such as Fourier transform infrared spectroscopy). For example, the nanoparticulate polymer or aggregate may show one or more absorbance bands in an infrared spectrum centred:
in a range of about 3680- about 2700 cm⁻¹;
in a range of about 1800- about 1200 cm⁻¹;
in a range of about 2330- about 2020 cm⁻¹;
in a range of about 1200- about 1010 cm⁻¹;
in a range of about 1010- about 700 cm⁻¹;
in a range of about 3600-3100 cm⁻¹;
in a range of about 3100-2700 cm⁻¹; and/or
a mixture thereof.

In another embodiment, the nanoP³ material, nanoparticulate polymer or aggregate is characterised using the zeta potential of the nanoparticulate polymer or aggregate. For example, the nanoP³ material, nanoparticulate polymer or aggregate may possess a zeta potential in a range from about −100 mV to about +100 mV. For example the zeta potential may be in a range from about −50 mV to about 60 mV.

In a further embodiment, the nanoP³ material, nanoparticulate polymer or aggregate has a zeta potential in a range of from about −80 mV to about +80 mV, when measured in a solution within a pH range of about 2 to about 10.

The nanoP³ material may have rough cauliflower like surface morphology. This may be due to their formation by aggregation of the nanoparticulate polymers. Thus the nanoP³ material may be aggregates of the nanoparticulate polymers. The nanoP³ material and aggregates may be spherical, or generally spherical. The nanoparticulate polymers may have a diameter of about 1 to about 50 nm, or about 5 to 10, 5 to 10, 10 to 50, 20 to 50 or 10 to 30 nm, e.g., about 5, 10, 15, 20, 25, 30, 35, 40, 45 or 50 nm. Aggregates (and the associated nanoparticulate polymers) may have embedded highly reactive radicals that diminish in quantity over time. The nanoP³ material may also have embedded long-lived and stable radicals in delocalised orbitals of carbon clusters. The stable radicals (secondary radicals) may result from reactions involving highly reactive radicals (primary radicals). The nanoP³ material may have a surface that is hydrophilic. The surface may therefore allow ready dispersion of the nanoP³ material in water. It may allow retention of bioactivity of surface immobilised bioactive molecules. The hydrophilic surface may be a result of oxidation of radicals during formation or after formation by exposure of the surface to air. Following conjugation with one or more second species, the nanoP³ material conjugate may be hydrophilic or hydrophobic. Alternatively, the nanoP³ material conjugate may display amphiphilic properties.

Depending on the monomers used or the conditions applied during the polymerisation, the nanoP³ material may be crosslinked. The term “crosslinked” herein refers to a polymer composition containing intramolecular and/or intermolecular bonds. These crosslinking bonds may be covalent or non-covalent in nature. Non-covalent bonding includes hydrogen bonding, electrostatic bonding, and ionic bonding.

One potential advantage obtained from crosslinking is the stability of the resulting nanoP³ material and potentially a conjugate formed from such a material. For example, crosslinking can reduce the solubility of the nanoP³ material (or a resulting conjugate) in comparison to similar compositions which are not crosslinked. In addition, the crosslinked nature of the nanoP³ material (or a conjugate formed thereof), may increase the chemical resistance of the nanoparticulate material or resulting conjugate.

The nanoP³ may be doped with additional inorganic elements or compounds, or organometallic compounds, which act as image enhancing contrast agents in medical imaging techniques. The nanoP³ may be doped with magnetic resonance imaging (MRI) contrast agents such as iron oxide or gadolinium compounds. Examples of imaging enhancing contrast agents include: fluorescent dyes (e.g., Alexa 680, indocyanine green, and Cy5.5); isotopes and radionuclides, such as: ¹¹C, ¹³N, ¹⁵O, ¹⁸F, ³²P, ⁵¹Mn, ^{52m}Mn, ⁵²Fe, ⁵⁵Co, ⁶²Cu, ⁶⁴Cu, ⁶⁷Cu, ⁶⁷Ga, ⁶⁸Ga, ⁷²As, ⁷³Se, ⁷⁵Br, ⁷⁶Br, ^{82m}Rb, ⁸³Sr, ⁸⁶Y, ⁹⁰Y, ⁸⁹Zr, ^{94m}Tc, ⁹⁴Tc, ⁹⁹mTc, ¹¹⁰In, ¹¹¹In, ¹²⁰I, ¹²³I, ¹²⁴I, ¹²⁵I, ¹³¹I, ¹⁵⁴Gd, ¹⁵⁵Gd, ¹⁵⁶Gd, ¹⁵⁷Gd, ¹⁵⁸Gd, ¹⁷⁷Lu, ¹⁸⁶Re, ¹⁸⁸Re, and ²²³Ra; paramagnetic ions, such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III); metals, such as lanthanum (III), gold (III), lead (II), and bismuth (III); oxides of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III); metals, such as lanthanum (III), gold (III), lead (II), and bismuth (III), including iron oxide and gadolinium oxide; ultrasound-contrast enhancing agents, such as liposomes; and radiopaque agents, such as barium, gallium, and thallium compounds. The imaging enhancing contrast agents may be may be incorporated directly onto the nanoP³ material, or indirectly by using an intermediary functional group, such as chelators.

In an exemplary process, the nanoP3 material may be prepared by plasma polymerisation, through the activation of a gaseous mixture of N₂/C₂H₂/Ar at 150 mTorr and application of 50 W of radiofrequency power.

### Monomer

The nanoP³ materials described herein are derived from one or more monomers.

In one embodiment the one or more monomers are used in a gaseous form for forming the nanoP³ material.

A monomer may be a hydrocarbon. Examples of hydrocarbons include alkenes, alkynes, cycloalkenes and cycloalkynes.

Examples of suitable alkene monomers include, but are not limited to: ethylene, propene, 1-butene, 1-pentene, 1-hexene, 1-heptene, 1-octene, 1-nonene, 1-decene, isomers thereof, or a mixture thereof.

Examples of suitable alkyne monomers include, but are not limited to: ethyne (acetylene), propyne, 1-butyne, 1-pentyne, 1-hexyne, 1-heptyne, 1-octyne, 1-nonyne 1-decyn, isomers thereof, or a mixture thereof.

Examples of suitable cycloalkene monomers include, but are not limited to: cyclopropene, cyclobutene, cyclopentene, cyclohexene, cycloheptene, cyclooctene, 1,3-cyclohexadiene, 1,4-cyclohexadiene, 1,5-cyclooctadiene, isomers thereof, or a mixture thereof.

Examples of suitable cycloalkyne monomers include, but are not limited to: cycloheptyne, cyclooctyne, cyclononyne, isomers thereof, or a mixture thereof.

In one embodiment an alkene is used as a monomer. The alkene may be the only monomer utilised in the formation of nanoparticulate polymer, or it may be used in the presence of at least one other monomer to form a copolymer, for example another alkene and/or an alkyne, cycloalkene or cycloalkyne.

In one embodiment an alkyne is used as a monomer. The alkyne may be the only monomer utilised in the formation of nanoparticulate polymer, or it may be used in the presence of at least one other monomer to form a copolymer, for example another alkyne and/or an alkene, cycloalkene or cycloalkyne. In a further embodiment acetylene is used as a monomer, either on its own or in the presence of at least one other monomer.

In another embodiment acetylene is used as a monomer in combination with at least one other monomer, for example at least one other monomer which is an alkene, alkyne, cycloalkenes or cycloalkyne.

In one embodiment a cycloalkene is used as a monomer. The cycloalkene may be the only monomer utilised in the formation of nanoparticulate polymer, or it may be used in the presence of at least one other monomer to form a copolymer, for example another cycloalkene and/or an alkene, alkyne or cycloalkyne.

In one embodiment a cycloalkyne is used as a monomer. The cycloalkyne may be the only monomer utilised in the formation of nanoparticulate polymer, or it may be used in the presence of at least one other monomer to form a copolymer, for example another cycloalkyne and/or an alkene, alkyne or cycloalkene.

Other monomers that may be used for forming the nanoP³ include perfluorocarbons, ethers, esters, amines, alcohols or carboxylic acids.

Examples of suitable perfluorocarbons include, but are not limited to: perfluoroallyl benzene.

Examples of suitable ethers include, but are not limited to: diethylene glycol vinyl ether, diethylene glycol divinyl ether, diethylene glycol monoallyl ether, or a mixture thereof.

Examples of suitable amines include, but are not limited to: allylamine, cyclopropylamine, poly (vinyl amine), or a mixture thereof.

Examples of suitable alcohols include, but are not limited to: poly (vinyl alcohol), allyl alcohol, ethanol, or a mixture thereof.

Examples of suitable carboxylic acids include, but are not limited to acrylic acid.

Biologically active agents

As used herein, the term “biologically active agent” refers to any agent (e.g. peptide, polypeptide, nucleic acid or small molecule drug) that has biological and/or pharmacological activity in vivo. A person skilled in the art would understand that biologically active agents or drugs that are known to prevent restenosis or prevent inflammation, or agents that treat cardiovascular disease may be a suitable agent to be conjugated to a nanoparticle as described herein. Examples of suitable biologically active agents include, but are not limited to, anti-platelets and anticoagulants, antithrombotic and fibrinolytic agents, anti-replicate and anti-proliferative agents, anti-inflammatory agents, cardiovascular agents, proteins, peptides and nucleotides.

As used herein, the term “peptide” is intended to mean any polymer comprising amino acids linked by peptide bonds. The term “peptide” is intended to include polymers that are assembled using a ribosome as well as polymers that are assembled by enzymes (i.e., non-ribosomal peptides) and polymers that are assembled synthetically. In various embodiments, the term “peptide” may be considered synonymous with “protein,” or “polypeptide”. In various embodiments, the term “peptide” may be limited to a polymer of greater than 50 amino acids, or alternatively, 50 or fewer amino acids. In various embodiments, the term “peptide” is intended to include only amino acids as monomeric units for the polymer, while in various embodiments, the term “peptide” includes additional components and/or modifications to the amino acid backbone. For example, in various embodiments, the term “peptide” may be applied to a core polymer of amino acids as well as derivatives of the core polymer, such as core polymers with pendant polyethylene glycol groups or core polymers with amide groups at the amino or carboxy terminus of the amino acid chain.

A “peptide mimetic” can be a molecule such as a peptide, a modified peptide or any other molecule that biologically mimics active ligands biomolecules such as enzyme substrates or cytokines. For example, a peptide mimetic may antagonise, stimulate, or otherwise modulate the physiological activity of a cytokine involved in the inflammatory process. Alternatively, the peptide mimetic mimics the activity of its natural protein in the treatment of cardiovascular disease.

Here the term “protein” refers to a sequence of amino acids for which the chain length is sufficient to produce the higher levels of tertiary and/or quaternary structure. The protein may have a molecular weight in a range of about 300 Da to about 150 kDa. The protein may have a molecular weight of greater than 150 kDa, or a molecular weight less than 300 Da.

### Anti-Platelets and Anticoagulants

Adhesion and platelet aggregation may be prevented by antiplatelets and anticoagulants. In one example, the biologically active agent is an antiplatelet agent. In another example, the biologically active agent is an anticoagulant.

Examples of suitable antiplatelets include, but are not limited to, aspirin and dipyridamole. Aspirin is classified as an analgesic, antipyretic, anti-inflammatory, antiplatelet drug. It has been clinically tested and proven to reduce the risk of sudden death and/or non-fatal reinfarction in post myocardial infarction (heart attack) patients. The proposed mechanism of how aspirin works, relates directly to the platelets. It somehow blocks the platelets, restricting coagulation. This prevents the cascading platelet aggregation found in thrombus and subsequently restenosis. Aspirin is therefore a possible restenosis inhibitor. Dipyridamole is a drug similar to aspirin, in that is has anti-platelet characteristics. Dipyridamole is also classified as a coronary vasodilator. It increases coronary blood flow by primary selective dilatation of the coronary arteries without altering systemic blood pressure or blood flow in peripheral arteries. These vasodilation characteristics are thought to be possibly beneficial for restenosis prevention.

Examples of suitable anticoagulant drugs include, but are not limited to, Heparin, Coumadin, Protamine and Hirudin. These drugs function as an anticoagulant by preventing the production of thrombin, a binding agent which causes blood to clot. This too, may reduce the cascading effect of platelet aggregation at the lesion site, thus possibly reducing restenosis. The use of Protamine in the presence of Heparin causes the Protamine to function as a Heparin antagonist, blocking the effect of the Heparin. Protamine, however, used alone, acts as an anticoagulant. Hirudin is singled out because it is not normally found in the human body. Hirudin is a drug that is found in the salivary glands of leeches. It is a very concentrated anticoagulant that functions in a similar manner as Heparin, Coumadin, and Protamine.

### Antithrombotic and Fibrinolytic Agents

In one example, the biologically active agent is an antithrombotic agent. In another example, the biologically active agent is an fibrolytnic agent.

Examples of antithrombotic and fibrinolytic agents include, but are not limited to, glycoprotein IIb/IIIa inhibitors, direct thrombin inhibitors, heparins, low molecular weight heparins, platelet adenosine diphosphate (ADP) receptor inhibitors, fibrinolytic agents (including streptokinase, urokinase, recombinant tissue plasminogen activator, reteplase and tenecteplase), enzymes (including: streptokinase, urokinase, tissue plasminogen activator (tPA), and plasmin), or a mixture thereof.

### Anti-Replicate and Anti-Proliferative Agents

There are several types of drugs that interrupt cell replication. Without wishing to be bound by theory, antimitotics (cytotoxic agents) work directly to prevent cell mitosis (replication), whereas antimetabolites prevent deoxyribose nucleic acid (DNA) synthesis, thus preventing replication. In one example, the biologically active agent is an anti-replicate drug. In one example, the biologically active agent is an anti-proliferative agent.

Examples of suitable anti-replicate drugs include, but are not limited to, Methotrexate, Colchicine, Azathioprine, Vincristine, VinBlastine, Fluorouracil, Adriamycin, and Mutamycin.

Examples of suitable anti-proliferative drugs include, but are not limited to, target of rapamycin (mTOR) inhibitors (including sirolimus, everolimus and ABT-578); paclitaxel and antineoplastic agents (including alkylating agents, e.g., cyclophosphamide, mechlorethamine, chlorambucil, melphalan, carmustine, lomustine, ifosfamide, procarbazine, dacarbazine, temozolomide, altretamine, cisplatin, carboplatin and oxaliplatin). In one embodiment, the biologically active agent is paclitaxel.

In another embodiment, the biologically active agent is sirolimus ((1R,9S,12S,15R,16E,18R,19R,21R,23S,24E,26E,28E,30S,32S,35R)-1,18-dihydroxy-12-[(2R)-1-[(1S,3R,4R)-4-hydroxy-3-methoxycyclohexyl]propan-2-yl]-19,30-dimethoxy-15,17,21,23,29,35-hexamethyl-11,36-dioxa-4-azatricyclo[30.3.1.0^{4.9}]hexatriaconta-16,24,26,28-tetraene-2,3,10,14,20-pentone; CAS no. 53123-88-9). Sirolimus, also known as rapamycin, is a macrolide compound believed to inhibit the activation of T cells and B cells by reducing their sensitivity to IL-2 by mTOR inhibition. Sirolimus is immunosuppressive drug, which has previously been reported as contraindicated with wound healing. Patients receiving this drug are often observed to have impaired surgical site wound healing. Areas of blood vessel treated with sirolimus are known to have delays in re-endothelialisation due to the anti-proliferative action of the drug. Sirolimus is shown as Formula I.

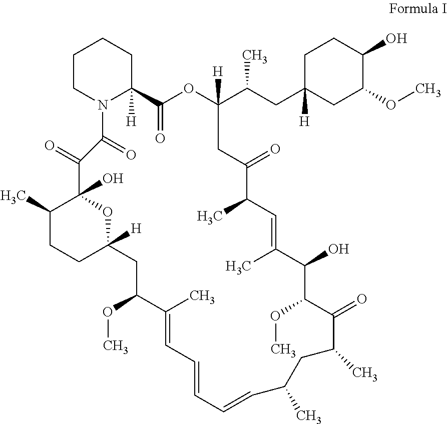

### Anti-Inflammatory Agents

Anti-inflammatory biologically active agents or drugs may also be useful to locally suppress inflammation caused by injury to luminal tissue during angioplasty. In one example, the biologically active agent is an anti-inflammatory agent. For example, the anti-inflammatory agent may be an anti-inflammatory drug or a biological molecule.

Suitable anti-inflammatory drugs include, but are not limited to, corticosteroids such as dexamethasone, betamethasone and prednisone and broad spectrum immunosuppressants such as Sulindac (2-[(3Z)-6-fluoro-2-methyl-3-[(4-methylsulfinylphenyl)methylidene]inden-1-yl]acetic acid; CAS no. 38194-50-2), Naproxen (CAS no. 22204-53-1) and aspirin (CAS no. 50-78-2). In one embodiment, the biologically active agent is Sulindac (2-[(3Z)-6-fluoro-2-methyl-3- [(4-methylsulfinylphenyl)methylidene]inden-1-yl]acetic acid; CAS no. 38194-50-2). Sulindac, shown as Formula II, is a non-steroidal anti-inflammatory drug.

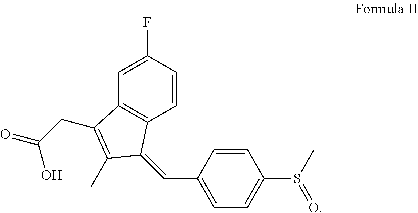 [SMILES: CC1=C(CC(=O)O)c2cc(F)ccc2/C1=C\c1ccc(S(C)=O)cc1]

Suitable anti-inflammatory agents include cytokines or fragments thereof. Cytokines are small proteins (˜5-20 KDa) that are important in cell signalling. Inflammation is characterized by an interplay between pro- and inflammatory cytokines. Cytokines may include chemokines, interferons, interleukins, lymphokines and tumour necrosis factors. Suitable examples include but are not limited to, anti-inflammatory cytokines such as IL4, IL-10, IL-13, IFN-alpha and transforming growth factor-beta.

It is known in the art that macrophage polarization may be alternatively activated by interleukins. The M1 phenotype is pro-inflammatory and the M2 phenotype is anti-inflammatory. Thus, it would be understood by a person skilled in the art that interleukins known to activate the M2 anti-inflammatory phenotype would be a suitable biologically active agent to conjugate to a nanoparticle as described herein. In one example, the biologically active agent is a cytokine or a fragment thereof. In one example, the biologically active agent is interleukin-4 (IL-4). In another example, the biologically active agent is interleukin-10 (IL-10).

### Cardiovascular Agents

In one example, the biologically active agent may be a pharmaceutical drug for the treating cardiovascular disease. Suitable pharmaceutical drugs include, but are not limited to, ACE inhibitors, antigotensin receptor blockers, calcium channel blockers, vasodilators and statins (also known as HMG-CoA reductase inhibitors). For example, the biologically active agent is a statin, such as simvastatin, pitavastatin, lovastatin, fluvastatin; or a mixture thereof. In one embodiment, the biologically active agent is simvastatin.

It is also contemplated that nitric oxide releasing agents may be a suitable agent to be conjugated to a nanoparticle as described herein.

### Antibodies

In one embodiment, the biologically active agent is an antibody. The antibody may be an antibody which can target the biologically active agent to the correct location in the blood vessel. Additionally or alternatively, the antibody may be an antagonist, for example a cytokine inhibitor.

The terms “antibody” or “antibodies” as used herein shall be taken to encompass a protein that comprises a variable region made up of one or more immunoglobulin chains, e.g., a polypeptide comprising a V_{L} and a polypeptide comprising a V_{H}. An antibody also may comprise constant domains, some of which can be arranged into a constant region or constant fragment or fragment crystallizable (Fc). A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ϵ, γ, or μ. The antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, de-immunized antibodies, non-depleting antibodies, non-activating antibodies, primatized antibodies, human antibodies and chimeric antibodies. As used herein, the term “antibody” is also intended to include formats other than full-length, intact or whole antibody molecules, such as Fab, F(ab′)2, and Fv which are capable of binding to an epitopic determinant. These formats may be referred to as antibody “fragments”. These antibody formats retain some ability to selectively bind to a target protein, examples of which include, but are not limited to, the following:
(1) Fab, the fragment which contains a monovalent binding fragment of an antibody molecule and which can be produced by digestion of whole antibody with the enzyme papain to yield an intact light chain and a portion of one heavy chain;
(2) Fab′, the fragment of an antibody molecule which can be obtained by treating whole antibody with pepsin, followed by reduction, to yield an intact light chain and a portion of the heavy chain; two Fab′ fragments are obtained per antibody molecule;
(3) (Fab′)₂, the fragment of the antibody that can be obtained by treating whole antibody with the enzyme pepsin without subsequent reduction; F(ab)2 is a dimer of two Fab′ fragments held together by two disulfide bonds;
(4) Fv, defined as a genetically engineered fragment containing the variable region of the light chain and the variable region of the heavy chain expressed as two chains;
(5) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc. which may or may not be polyspecific; and
(6) Single domain antibody, typically a variable heavy domain devoid of a light chain.

Accordingly, an antibody as described herein may include separate heavy chains, light chains, Fab, Fab′, F(ab′)2, Fc, a variable light domain devoid of any heavy chain, a variable heavy domain devoid of a light chain and Fv. Such fragments can be produced by recombinant DNA techniques, or by enzymatic or chemical separation of intact immunoglobulins. Any of the antibodies or fragments thereof described herein and otherwise known in the art can be conjugated to the nanoP³ particles disclosed herein.

Thus, the biologically active agent may be an antibody or a protein or peptide which targets a factor involved in the inflammation process.

Herein the term “targeting ligand” refers to a molecule that binds to or interacts with a target molecule. Typically the nature of the interaction or binding is non-covalent, e.g., by hydrogen, electrostatic, or van der Waals interactions, however, binding may also be covalent.

The term “ligand”, as used herein, refers to compounds which target biological markers. Examples of ligands include, but are not limited to, proteins, peptides, antibodies, antibody fragments, saccharides, carbohydrates, glycans, cytokines, chemokines, nucleotides, lectins, lipids, receptors, steroids, neurotransmitters, Cluster Designation/Differentiation (CD) markers, imprinted polymers, and the like.

Examples of targeting ligands include, but are not limited to, a nuclear localisation signal (for example KR[PAATKKAGQA]KKKK), RGD, NGR, Folate, Transferrin, GM-CSF, galactosamine, anti-VEGFR, anti-ERBB2, anti-CD20, anti-CD22, anti-CD19, anti-CD33, anti-CD25, anti-tenascin, anti-CEA, anti-MUC1, anti-TAG72, anti-HLA-DR10, or a mixture thereof.

### Polynucleotides

The biologically active agent may be a polynucleotide that can modulate the inflammation process. The term “polynucleotide” as used herein shall be taken to encompass DNA, RNA, an antisense polynucleotide, a ribozyme, an interfering RNA, a siRNA, a microRNA, and any other polynucleotide known in the art. The polynucleotide may encode a protein or a functional RNA (such as an interfering RNA) which can disrupt inflammation. Thus, the polynucleotide may be a polynucleotide vector or plasmid.

Examples of gene targeting agents include, but are not limited to, DNA (gDNA, cDNA), RNA (sense RNAs, antisense RNAs, mRNAs, tRNAs, rRNAs, small interfering RNAs (siRNAs), short hairpin RNAs (ShRNAs), micro RNAs (miRNAs), small nucleolar RNAs (SnoRNAs, small nuclear (snRNAs)) ribozymes, aptamers, DNAzymes, antisense oliogonucleotides, vectors, plasmids, other ribonuclease-type complexes, and mixtures thereof. For example, the biologically active agent may be an siRNA against the p65 subunit of NF-κB, thus modifying NF-κB mediated inflammation or a gene targeting agent for IkB kinase or AP-1, which encode cytokine expression and/or cytokine receptor expression

### Stem Cells

The biologically active agent may be a stem cell, for example skeletal myoblasts, bone-marrow derived stem cells, bone-marrow derived mononuclear cells, bone-marrow derived hematopoietic stem cells and endothelial progenitor cells, mesenchymal stromal/stem cells, cardiac stem and progenitor cells, induced pluripotent stem cells or a mixture thereof. For example, the stem cell may be a mesenchymal stromal/stem cell which adopt an immune-suppressive phenotype in the presence of pro-inflammatory cytokines.

### Imaging Agents

In one embodiment, the agent is an imaging agent. Imaging agents may be used in vivo to study vascular structure, function and angiogenesis. Examples of suitable imaging agents include, but are not limited to, luciferase; fluorescently labelled dyes and antibodies, contrast agents (including: iopamidol, iohexol and ioxilan); barium sulfate; Indocyanine green (ICG), and mixture thereof.

The imaging agent may be an imaging enhancing contrast agent. Examples of suitable imaging enhancing contrast agents include but are not limited to, fluorescent dyes (e.g., Alexa 680, indocyanine green, and Cy5.5); isotopes and radionuclides, such as: ¹¹C, ¹³n, ¹⁵O, ¹⁸F, ³²P, ⁵¹Mn, ^{52m}Mn, ⁵²Fe, ⁵⁵Co, ⁶²Cu, ⁶⁴Cu, ⁶⁷Cu, ⁶⁷Ga, ⁶⁸Ga, ⁷²As, ⁷³Se, ⁷⁵Br, ⁷⁶Br, ^{82m}Rb, ⁸³Sr, ⁸⁶Y, ⁹⁰Y, ⁸⁹Zr, ^{94m}Tc, ⁹⁴Tc, ⁹⁹mTc, ¹¹⁰In, ¹¹¹In, ¹²⁰I, ¹²³I, ¹²⁴I, ¹²⁵I, ¹³¹I, ¹⁵⁴Gd, ¹⁵⁵Gd, ¹⁵⁶Gd, ¹⁵⁷Gd, ¹⁵⁸Gd, ¹⁷⁷Lu, ¹⁸⁶Re, ¹⁸⁸Re, and ²²³Ra; paramagnetic ions, such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III); metals, such as lanthanum (III), gold (III), lead (II), and bismuth (III); oxides of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III); metals, such as lanthanum (III), gold (III), lead (II), and bismuth (III), including iron oxide and gadolinium oxide; ultrasound-contrast enhancing agents, such as liposomes; and radiopaque agents, such as barium, gallium, and thallium compounds. The imaging enhancing contrast agents may be may be conjugated directly onto the nanoP³ material, or indirectly by using an intermediary functional group, such as chelators.

### Conjugates

Herein the term “conjugate” refers to molecules formed by the attachment of one or more compounds to a nanoparticulate polymer or an aggregate comprising nanoparticulate polymers. The “one or more compounds” may be a biologically active agent as defined herein. The attachment may be via a covalent bond or an electrostatic interaction.

The nanoparticulate polymers, aggregates or nanoP³ materials described herein are conjugated to an agent. The agent may be a biologically active agent or an imaging agent. In one embodiment, the agent is a biologically active agent. In another embodiment, the agent is an imaging agent. The binding of the agent may be modulated by changing the pH of the reaction conditions during the conjugation process. The pH of the solution modulates the charge of nanoP³ through protonation or de-protonation of surface functional groups, such as amines and carboxylic acid groups. For example, the binding of positively-charged conjugates to nanoP³ materials may be improved by increasing the pH of the solution containing the nanoP³ materials and the agent. At highly alkaline media, nanoP3 becomes becomes negatively charged via deprotonation carboxylic surface groups, which also stabilizes the nanoparticles due to the repulsion of between negatively charged particles. Alternatively, the binding of negatively-charged conjugates to nanoP³ materials may be improved by decreasing the pH of the solution containing the nanoP³ materials and the agent.

Disclosed herein in the use of a nanoP³ material (for example: a nanoparticle polymer, an aggregate, or a mixture thereof), in the formation of a conjugate.

The conjugate may comprise only a single agent. Alternatively the conjugate may comprise two or more different agents, for example two, three or four second agents.

The nanoP³ materials are generally capable of directly coupling, for example by covalent coupling or ionic coupling, to an agent, for example a biologically active agent or an imaging agent. When the nanoparticle polymer or aggregate is a plasma polymer, the coupling process is generally rapid and capable of proceeding under mild conditions. The coupling may be by means of unpaired electrons in the polymer structure (i.e., radical sites) or by means of functional groups generated on the nanoP³ material, or incorporated onto the resulting conjugate via the addition of an appropriate biologically active agent or by reaction with air (or another gas), or some other fluid to which the nanoP³ material is exposed. The nanoP³ material may include monomer units which comprise functional moieties which are capable of chemically coupling an agent. The conjugation of one or more agents with the nanoP³ material may introduce functional groups on or in the resulting conjugate, which can be used for further chemical reactions or used as binding sites for processes such as biochemical/biological processes taking place in in vitro or in vivo conditions.

Herein, functional moieties (or “functional groups”), refers to a group of atoms present on the monomer, nanoP³ material, or conjugate comprising a nanoP³ material, which can react with other complimentary functional groups, for example other functional groups present on an agent. Functional groups include but are not limited to, the following moieties: carboxylic acid (—(C═O)OH), carbonyl, primary or secondary amine (—NH₂, —NH—), nitric oxide, maleimide, thiol (—SH), sulfonic acid (—(O═S═O)OH), carbonate, carbamate (—O(C═O)N<), hydroxy (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, vinyl sulfone, vinyl ketone, diazoalkane, oxirane, and aziridine, or a mixture thereof.

Accordingly, in one aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 5 nm to about 500 nm;
and a biologically active agent selected from the group consisting of: an anti-inflammatory cytokine; anti-inflammatory drug; a statin drug and an anti-proliferative drug.

In one embodiment, the aggregate has a mean diameter in a range of about 5 to 500 nm, or about 5 to about 400 nm, or about 5 to about 300 nm, or about 5 to about 200 nm, or about 5 to about 100 nm, or about 50 to about 100 nm, or about 100 to about 500 nm, or about 150 to about 500 nm, or about 180 nm to about 500 nm, or about 100 to about 400 nm, or about 150 to about 400 nm, or about 180 to about 400 nm, or about 100 to about 300 nm, or about 150 to 300 nm, or about 180 to 300 nm, or about 100 to about 200 nm, or about 150 to about 200 nm, or about 180 to about 200 nm, or about 150 to about 250 nm, or about 180 to about 250 nm, or about 200 to about 400 nm, or about 200 nm to about 300 nm, or a mixture thereof. In one embodiment, the aggregate has a mean diameter of about 200 nm. In one embodiment, the aggregate has a mean diameter of about 100 nm. In one embodiment, the aggregate has a mean diameter of about 100 nm to about 200 nm. In another embodiment, the aggregate has a mean diameter of about 50 to about 100 nm.

In one embodiment, the anti-inflammatory cytokine is IL-4. The IL-4 may be bound to the nanoP3 at a loading capacity of about 0.5 μg, about 0.6 μg, about 0.7 μg, about 0.8 μg, about 0.9 μg, about 1.0 μg or about 1.1 μg IL-4/10⁹ nanoP3, or equivalent concentrations at alternative amounts of nanoP3.

In one embodiment, the anti-inflammatory cytokine is IL-10. The IL-10 may be bound to the nanoP3 at a loading capacity of about 0.5 μg, about 0.6 μg, about 0.7 μg, about 0.8 μg, about 0.9 μg, about 1 μg, about 1.1 μg, about 1.2 μg, about 1.3 μg or about 1.4 μg IL-10/ 10⁹ nanoP3, or equivalent concentrations at alternative amounts of nanoP3. In one example, the IL10 may be bound to the nanoP3 at a loading capacity of about 1.3 μg IL-10/10⁹ nanoP3, or equivalent concentrations at alternative amounts of nanoP3.

Accordingly in another aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm;
and IL-10. Alternative mean diameters can be used as described herein.

In one embodiment, the anti-proliferative drug is Sirolimus. The Sirolimus may be bound to the nanoP3 at a loading capacity of about 1.5 μg to about 3 μg, or about 1.5 μg to about 2.5 μg, or about 2 μg to about 2.5 μg, or about 2 μg to about 3 μg Sirolimus/10⁹ nanoP3, or equivalent concentrations at alternative amounts of nanoP3. In one example, the Sirolimus may be bound to the nanoP3 at a loading capacity of about 2.50 μg Sirolimus/10⁹ nanoP3, or equivalent concentrations at alternative amounts of nanoP3.

The inventors have surprisingly found that the Sirolimus bound to nanoP3 increased re-endothelialisation, thus promoting vessel healing. This finding is contrary to the action of free Sirolimus.

Accordingly in another aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm;
and Sirolimus. Alternative mean diameters can be used as described herein.

In one embodiment, the anti-inflammatory drug is Sulindac. The Sulindac may be bound to the nanoP3 at a loading capacity of about 2 μg to about 3.5 μg, or about 2.5 μg to about 3.5 μg, or about 2.5 μg to about 3.1 μg Sulindac/10⁹ nanoP3, or equivalent concentrations at alternative amounts of nanoP3. In one example, the Sulindac may be bound to the nanoP3 at a loading capacity of about 3.05 μg sulindac/10⁹ nanoP3, or equivalent concentrations at alternative amounts of nanoP3.

Accordingly, in another aspect, the present disclosure provides a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm;
and sulindac. Alternative mean diameters can be used as described herein.

It is contemplated that the nanoparticulate polymers, aggregates or conjugates thereof could be incorporated into an appropriate scaffold for the treatment of cuts/wounds or vascular injury. For example, the nanoparticulate polymers, aggregates of conjugates thereof, may be used on or in implants, such as cardiac patches, vascular grafts and stents.

### Production of Conjugates

Details of the production of nanoP3 materials and nanoP3 conjugates are described at page 49, line 18 to page 71, line 7 of PCT Publication no. WO2018/112543, which is herein incorporated by reference. An exemplary process for the production of conjugates is provided below.

NanoP3 may be synthesized by plasma polymerisation, through activation of a gaseous mixture of N₂/C₂H2/Ar at 150 mTorr and by application of 50 W of radiofrequency power. The loading capacity and binding efficiency of nanoP3 to biologically active agents such as IL-4 and IL-10 may be measured using a fluorescently labelled molecule such as Cy5 to establish optimal incubation parameters for both in vitro and in vivo applications.

The biologically active agent may be mixed with the nanoP3 in ultrapure water in a total reaction volume of 1 ml and left to incubate for a 1 h at room temperature. Binding kinetics may be performed on a Clariostar monochromator microplate reader (BMG Labtech, Germany) using the leftover washes following 1 h incubations.

### Pharmaceutical Compositions

The conjugates may be present in a pharmaceutical composition. Details of suitable pharmaceutical compositions and methods for deriving suitable pharmaceutical compositions are described at page 40, line 7 to page 44, line 26 of PCT Publication no. WO2018/112543, which is herein incorporated by reference.

### Delivery of the Conjugate to Blood Vessels

Disclosed herein are methods of localizing a biologically active agent to a region of a blood vessel, methods of regulating inflammation to a region of a blood vessel and methods of retaining a biologically active agent to a site of delivery in a blood vessel by conjugating the biologically active agent to a nanoparticle, thereby producing a conjugate and delivering the conjugate to the region of the blood vessel.

The conjugate may be delivered to the blood vessel by any suitable method of delivery known in the art. Examples of suitable methods of delivery include, but are not limited to, a catheter, a stent, a stent graft, a graft and a valve or by direct injection into blood vessel.

In one example, the catheter is an occlusion perfusion catheter. In another example, the catheter is a sweating balloon catheter.

After delivery, the conjugate is retained at the site of delivery in the blood vessel. In one embodiment, the conjugate is retained in the region of the blood vessel for a period greater than the period unconjugated biologically active agent would be retained in the region of the blood vessel. For example, the conjugate is retained in the region of the blood vessel for at least 1.2, 1.2, 1.5, 1.7 or at least 2 times the period the unconjugated biological agent would be retained in the region of the blood vessel or the conjugate may be retained at the site of delivery for at least 1 day, or at least 2 days, or at least 3 days, or at least 4 days, or at least 5 days, or least 6 days, or least 7 days, or least 8 days, or least 9 days, or least 10 days, or least 11 days, or least 12 days, or least 13 days, or least 14 days. In one example the conjugate is retained at the site of delivery for at least 1 day. In another example, the conjugate is retained at the site of delivery for at least 5 days. In another example, the conjugate is retained at the site of delivery for at least 14 days.

### Methods of Treatment

Also disclosed herein, are methods treating or preventing vascular injury or vascular disease, the method comprising a step of delivering a conjugate, as defined herein, or a pharmaceutical composition comprising the conjugate as defined herein, to the subject. The term “treating” is used herein to encompass both therapeutic and prophylactic treatment. Thus, the methods of treatment disclosed herein may encompass methods of preventing one or more symptoms of a disease, disorder, or condition. It will be appreciated that “treating” may be interpreted as effecting a reduction in any one or more symptoms of a disease, disorder, or condition. Accordingly, “treating” encompasses a reduction in vascular occlusion or neointima formation and/or an increase in the rate of re-endothelialisation relative to a patient who has not received a conjugate disclosed herein or has received only an unconjugated biologically active agent. As used herein, “promoting healing” refers to an increase in any indicative marker of healing relative to a patient who has not received a conjugate disclosed herein or has received only an unconjugated biologically active agent. “Healing” may refer to the healing of a wound in a blood vessel. It will be appreciated that the term “healing” encompasses processes such as endothelialisation. Therefore, any reference hereinto promoting healing is to be understood as referring to the promotion of endothelialisation.

In one aspect, the present disclosure provides a method of treating or preventing vascular injury or vascular disease comprising delivering a conjugate as defined herein to a region of a blood vessel in a patient in need thereof.

Thus, in one example, the present disclosure provides a method of treating or preventing vascular injury or vascular disease comprising delivering a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm; and
i) interleukin-10; or
ii) Sulindac; or
iii) Sirolimus.

In another aspect, the present disclosure provides a method of promoting healing comprising delivering a conjugate as defined herein to a region of a blood vessel in a patient in need thereof.

Thus, in one example, the present disclosure provides a method of promoting healing comprising delivering a conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm; and
i) interleukin-10; or
ii) Sirolimus.

Also disclosed herein is the use of a conjugate as defined herein, in the manufacture of a medicament for treating or preventing a condition in a patient.

In one aspect, there present disclosure provides the use of a conjugate defined herein in the manufacture of a medicament for treating or preventing vascular injury or vascular disease in a patient in need thereof.

In one aspect, there present disclosure provides the use of a conjugate defined herein in the manufacture of a medicament for promoting healing in a patient in need thereof.

In one embodiment a conjugate as defined herein is used as a medicament or utilised in the manufacture of a medicament.

In another embodiment, a conjugate as defined herein is for use in treating or preventing a vascular disease.

In another embodiment, a conjugate as defined herein is for use in promoting healing.

In another aspect, the present disclosure provides a conjugate as defined herein when used for treating or preventing a vascular disease.

In another aspect, the present disclosure provides a conjugate as defined herein when used for promoting healing.

The conjugates, may be provided in an “effective amount”, for example when an appropriate compound is added to a pharmaceutical composition. The phrase “effective amount” is taken to mean an amount of the conjugate that will elicit a desired biological or medical response of a tissue, system, animal or human that is being sought by the researcher, veterinarian, medical doctor or other clinician administering the compound of a composition comprising the compound.

The “effective amount” will be dependent on a number of factors, including the efficacy of a particular conjugate. The patient's weight and age may also be a factor for the person skilled in the art when determining the concentration of compound that the patient should receive.

The phrases “administration of” and or “administering a” compound should be understood to mean providing a conjugate, or a pharmaceutical composition comprising a conjugate as defined herein, to a patient in need of treatment.

The recipients of a conjugate defined herein, can be human beings, male or female.

Alternatively the recipients of: a nanoparticulate, aggregate or conjugate; or a pharmaceutical composition comprising a nanoparticulate, aggregate or conjugate, can also be a non-human animal. “Non-human animals” or “non-human animal” is directed to the kingdom Animalia, excluding humans, and includes both vertebrates and invertebrates, male or female, and comprises: warm blooded animals, including mammals (comprising but not limited to primates, dogs, cats, cattle, pigs, sheep, goats, rats, guinea pigs, horses, or other bovine, ovine, equine, canine, feline, rodent or murine species), birds, insects, reptiles, fish and amphibians.

The recipients of the conjugates and pharmaceutically acceptable compositions are referred herein with the interchangeable terms “patient”, “recipient” “individual”, and “subject”. These four terms are used interchangeably and refer to any human or animal (unless indicated otherwise), as defined herein. The patient may be receiving or may have received an endovascular intervention. In one example, the patient is receiving an endovascular intervention. In another example, the patient has received an endovascular intervention. The vascular injury may be due to an endovascular intervention. For example, the endovascular intervention may be stenting or balloon angioplasty. In one example, the endovascular intervention may be stenting. In another example, the endovascular intervention is balloon angioplasty.

Diseases, disorders or conditions that could be treated using a conjugate described herein, include, but are not limited to, vascular diseases such as artherosclerosis, peripheral artery disease, acute coronary syndrome; gastrointestinal disease and renal disease.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### EXAMPLES

### Example 1

### Interleukin Surface Binding to NanoP3

Carrier free recombinant rat IL-4 and IL-10 (R&D systems, USA) were dissolved in phosphate buffered saline (PBS) at a stock concentration of 100 ng/μl. To establish binding kinetics, each interleukin was tagged with a Lightning-link Cy5 antibody label (Novus Biologicals, USA). For every 1×10⁹ nanoP3 (200 nm diameter) in ultrapure water (Thermofisher, USA), either 1.42 μg of IL-4 or 1.32 μg of IL-10 was added. Additional water was added to reach a total reaction volume of 1 ml and left to incubate for a 1 h at room temperature. Binding kinetics were performed on a Clariostar monochromator microplate reader (BMG Labtech, Germany) using the leftover washes following 1 h incubations.

The binding efficiency of nanoP3 to IL-4 in solution was maximum at 99%, corresponding to a loading capacity of 0.5±0.01 μg/10⁹ particles. The loading capacity was further increased to 1.1±0.02 μg/10⁹particles but at a lower binding efficiency of 53.1%. The maximum binding efficiency of nanoP3 to IL-10 was 99.9%, representing a total mass loading capacity of 0.5±0.02 μg/10⁹ particles. Further loading of IL-10 onto nanoP3 was observed up to 0.80±0.02 μg/10⁹ particles corresponding to a binding efficiency of 40%. All experiments were performed in ultra-pure water (pH=6.5) at room temperature. The incubation time was 30 minutes.

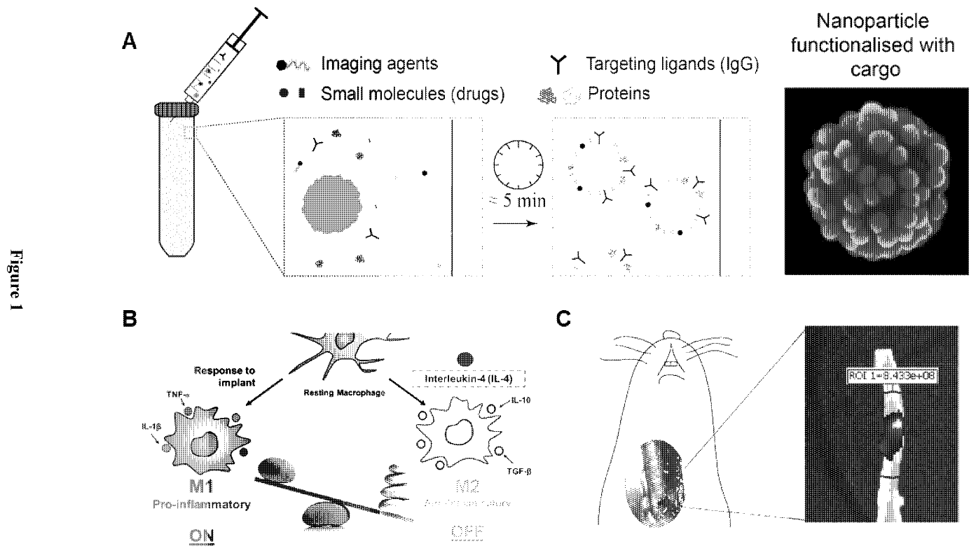

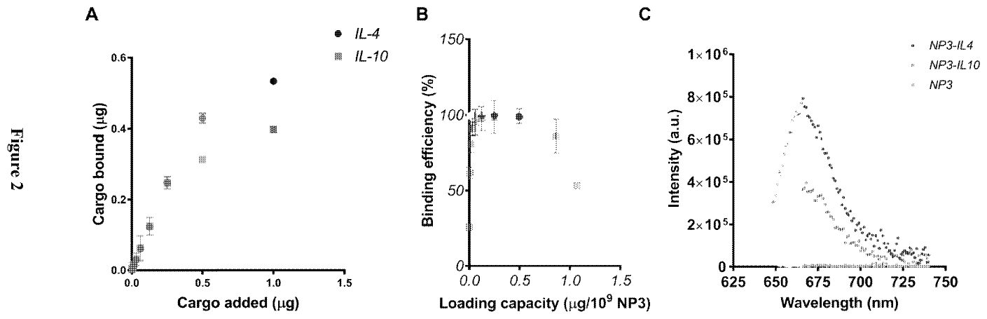

FIG. 1A is a schematic of the functionalisation of nanoP3. Small molecules, imaging agents, targeting ligands or proteins are incubated with nanoP3 to functionalise the nanoP3.

FIG. 1B shows the response of macrophages to IL-4 and IL-10. IL-4 and IL-10 can shift the phenotype of M1 macrophages (pro-inflammatory) to M2 macrophages (anti-inflammatory).

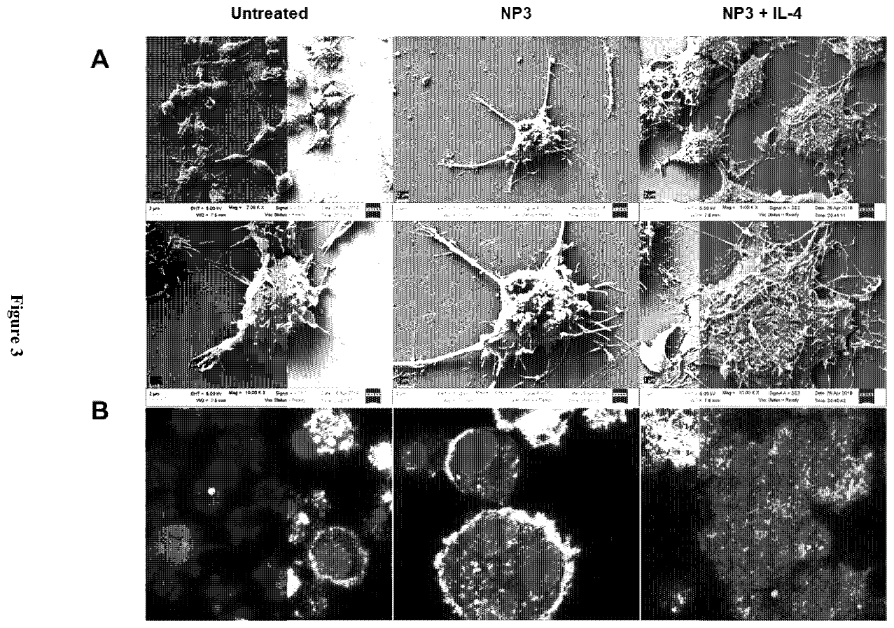

FIG. 2 shows the loading capacity of IL-4 and IL-10 on nanoP3. The binding efficiency of IL-4 and IL-10 is 100%. The emission spectrum confirms that nanoP3 can bind to IL-4 and IL-10.

### Example 2

### IL-4 Bound to NanoP3 Causes M2 Macrophage Polarisation

Directing the phenotype of macrophages to their M2 anti-inflammatory state has the potential to mitigate the further progression of vessel injury and promote disease regression. Various cytokines from the interleukin-family facilitate this shift in phenotype from M1 to M2, including IL-4 and IL-10.

The inventors sought to investigate the effect of NP3+IL-4 on macrophage polarisation in vitro. Raw 246.7 mouse macrophage cells (ATCC, USA) were cultured at 5×10³ cells/well in a 96 well plate. IL-4 bound nanoP3 was added into macrophage cultures at a concentration of 1×10⁵ nanoP3/well. After 24 hours, macrophages were fixed in 4% paraformaldehyde prior to scanning electron microscopy (SEM) and confocal imaging. Confocal staining was performed using an Actin cytoskeletal stain (Abcam, USA) and an anti-arginase-1 antibody (Abcam, USA).

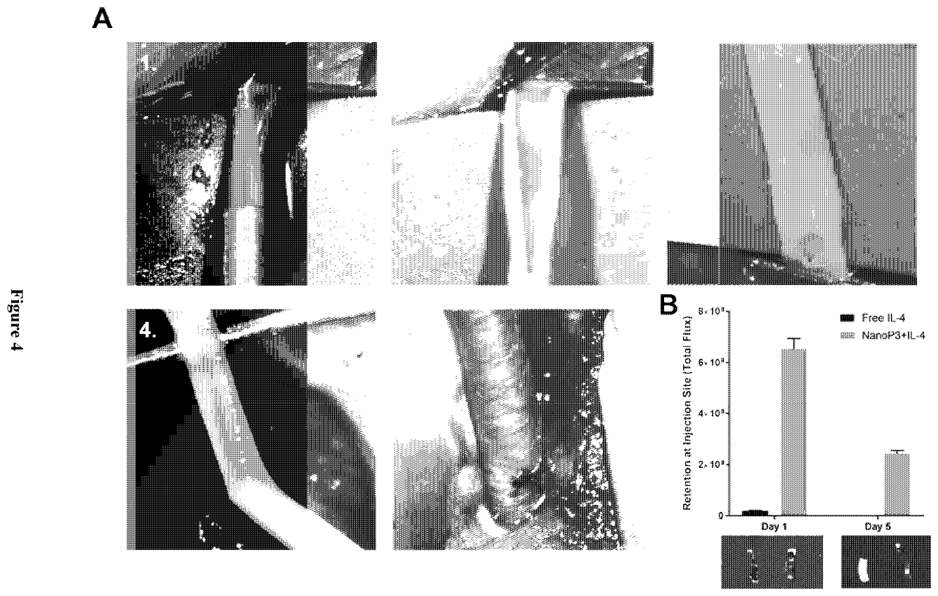

FIG. 3 shows that compared to untreated macrophages and macrophages that have been treated with only nanoP3, nanoP3-IL4 causes M2 activation. Confocal staining confirms that ARG-1, a highly expressed M2 enzyme is significantly upregulated in NP3-IL4 treated macrophages, further confirming M2 activation.

### Example 3

### Rat Carotid Injury and NanoP3 Delivery Model (I)

Using a rat carotid model of vessel injury, the efficacy of NP3 bound interleukins to treat cardiovascular pathology was determined in vivo (FIG. 1C).

Study approval was obtained from the Sydney Local Health District Animal Welfare Committee (protocol number 2017/006). Experiments were conducted in accordance with the Australian Code of Practice for the Care and Use of Animals for Scientific Purpose. Rats (Sprague Dawley, male, 7 weeks) were purchased from Laboratory Animal Service (NSW, Australia). Rats were given a single intramuscular injection of ketamine (75 mg/kg) and medetomidine (0.5 mg/kg) to induce anaesthesia. The common carotid artery was isolated and double ligations were spaced roughly 1 cm apart. A small incision at the distal end was made, through which microintraocular forceps (World Precision Instruments, USA) were inserted, expanded to their full width and rotated 360 degrees to injured the entire luminal surface area of the vessel. This was repeated 5 times before withdrawing the forceps, and inserting a 22G catheter into the same incision. Through the catheter, IL-4 or IL-10 bound nanoP3 solutions were injected at a concentration of 2×10⁸ nanoP3 in a total volume of roughly 80 μl of RPMI media. The nanoP3 solutions were allowed to incubate for 2 minutes and then withdrawn completely from the vessel. The incision was sutured closed using a 9-0 nylon suture, and both ligations were undone to re-establish blood flow. The isolated vessel segments were then explanted 14 days later for pathological evaluation.

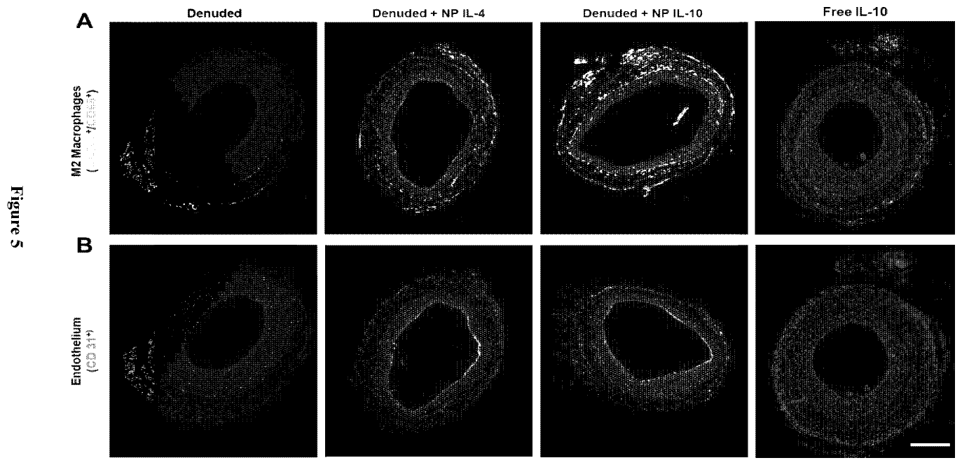

FIG. 4B shows that free IL-4 is immediately washed away from the vessel wall once blood flow is restored. However, IL-4 bound to nanoP3 is significantly retained in the vessel and persists at substantial levels after 5 days.

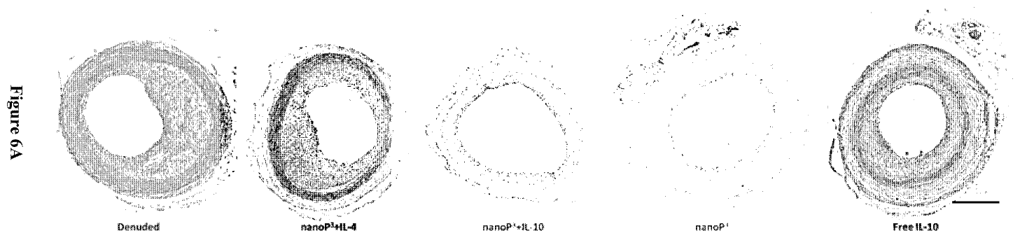

FIG. 5 shows that formation of neointima is inhibited. Immunostaining of the presence of M2 macrophages (yellow/green) in treated carotid segments shows a significant increase in NP3+IL-10 groups compared to denuded, NP3+IL-4, and free IL-10. Evaluation of repair of the damaged endothelium by immunostaining shows that both NP3+IL4 and NP3+IL-10 restore full endothelium integrity by 14 days after injury. However, this is absent when treated with free IL-10.

After 14 days in vivo, vessel explants were fixed in 4% PFA overnight and subjected to ethanol dehydrations prior to paraffin embedding. Embedded vessel segments were then sectioned in 5 μm thick slices in the longitudinal direction. M2 macrophage and luminal endothelium staining was conducted using anti-CD206 (Abcam, USA) and anti-VonWillebrand Factor (Sigma, USA) antibodies. Fluorescent imaging was conducted by using Alexa-fluor 594 secondary antibodies. Staining of neointima formation was conducted using hematoxylin & eosin (H&E) staining. Normalised hyperplasia was calculated as the total area of hyperplasia divided the area of the original vessel lumen.

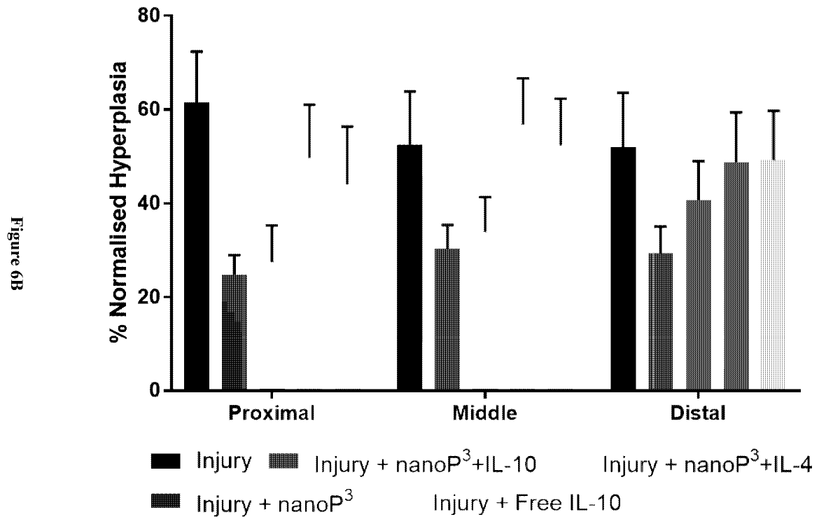

The analysis of two-week neointima formation following therapeutic nanoP3 delivery shows that vascular occlusion is reduced to approximately 35% and 20% of the cross-sectional lumen area in NP3+IL-4 and NP3+IL10 groups, respectively. Free IL-10 and nanoP3 alone has no significant effects on vascular occlusion, suggesting that the nanoP3 platform facilitates the therapeutic benefits of IL-10 (FIG. 6).

### Example 4

### Rat Carotid Injury and NanoP3 Delivery Model (II)

Using a rat carotid model of vessel injury, the efficacy of NP3+IL-10, NP3+Sirolimus or NP3+Sulindac to treat cardiovascular pathology was determined in vivo. Carrier -free recombinant rat IL-10 (1.34 μg/10⁹ nanoP3; R&D systems, USA), Sirolimus (2.50 μg/10⁹ nanoP3; rapamycin, Sigma-Merck, USA), Sulindac (3.05 μg/10⁹nanoP3; Sigma-Merck, USA), or a Cy7 fluorescent tag (5.03 μg/10⁹ nanoP3; CF750 Antibody Label, Sigma-Merck, USA) were diluted in sterile water and conjugated to 2×10⁹ nanoP3.

The rats were subjected to vessel injury as described in Example 3 except that through the catheter, IL-10, Sirolimus or Sulindac bound nanoP3 solutions were injected at a concentration of 2×10⁸ nanoP3 (IL-10, 0.268 μg; Sirolimus, 0.5 μg; Sulindac 0.61 μg; Cy7, 1.01 μg), in a total volume of roughly 80 μl of RPMI media.

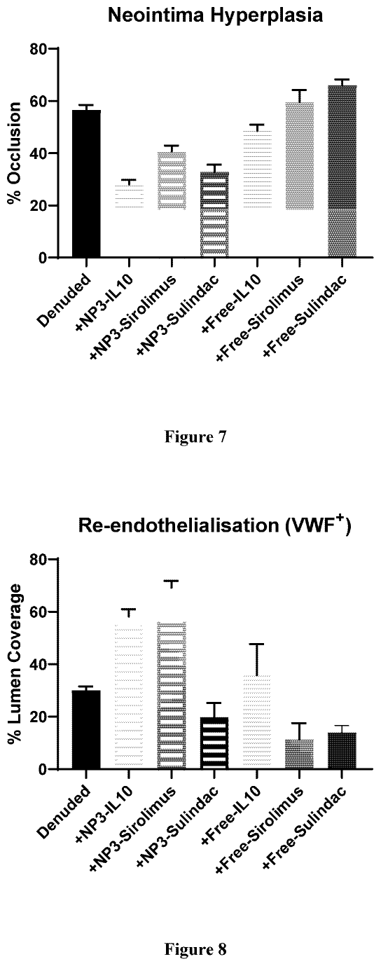

FIG. 7 shows that the delivery of NP3+IL-10, NP3+Sirolimus or NP3+Sulindac result in the inhibition of neointimal hyperplasia compared to the delivery of free agents in a rat carotid injury model.

Vessel re-endothelialisation in the rat carotid model treated with NP3+IL-10, NP3+Sirolimus or NP3+Sulindac was also investigated using von Willebrand factor (vwf) staining as described in Example 3. Sirolimus is an agent that is known to impair wound healing. Surprisingly, the inventors also show that, although Sirolimus has previously been described as impairing wound healing, both NP3+Sirolimus and NP3+IL10 stimulate healing (endothelialisation) (FIG. 8).

### Example 5

### IL-10 and Sulindac Bound to NanoP3 Cause M2 Macrophage Polarisation

The inventors sought to investigate the effect of NP3+IL-10 and NP3+Sulindac on macrophage polarisation in vitro.

Raw 246.7 mouse macrophage cells (ATCC, USA) were cultured at 5×10³ cells/well in a 96 well plate. IL-10 (1.34 μg/10⁹ nanoP3) and Sulindac (3.05μg/10⁹nanoP3) were bound to nanoP3 as described in Example 4.

NP3+IL-10 or NP3+Sulindac was added into macrophage cultures at a concentration of 1 x 10⁵ nanoP3/well. After 24 hours, macrophages were fixed in 4% paraformaldehyde prior to scanning electron microscopy (SEM) and confocal imaging. Confocal staining was performed using an Actin cytoskeletal stain (Abcam, USA) and an anti-arginase-1 antibody (Abcam, USA).

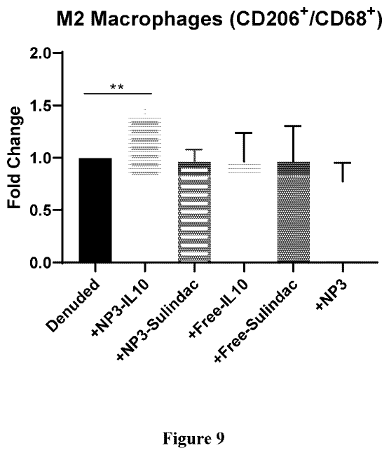

FIG. 9 shows that, compared to untreated macrophages (“denuded”) and macrophages that have been treated with only nanoP3 (“+NP3”), NP3+IL-10 causes M2 activation. Confocal staining confirms that ARG-1, a highly expressed M2 enzyme, is significantly upregulated in NP3+IL10 treated macrophages, further confirming M2 activation.

### Example 6

### Rabbit Iliac Injury and NanoP3 Delivery Model

Using a rabbit iliac injury model, the efficacy of NP3+IL-10 treatment was evaluated.

Study approval was obtained from the University of Sydney Animal Ethics Committee (AEC) protocol 2019-1653. Experiments were conducted in accordance with the Australian Code of Practice for the Care and Use of Animals for Scientific Purpose. An incision (˜1cm length) was made in the middle of the inguinal skin and the right femoral artery exposed after the blunt dissection of the muscle layers. The artery was ligated on the distal end and above the ligation, a small incision made to allow a 5F sheath (Abbott, TREK Coronary Dilatation Catheter, 3.25 mm) to be inserted. The sheath was held in place with sutures (3-0, silk). A 0.014 inch guide wire was inserted through the sheath, retrograde into the abdominal aorta. A 3.25 mm angioplasty balloon catheter was then inserted over the guide wire and advanced into the aorta. The location of NP3 delivery and the surrounding vasculature was determined by intravenous injection of contrast fluid ISOVUE 370 or OMNIPAQUE (75.5 g/100 ml), 25%-50% (v/v) contrast/saline prior to angiography.

Denudation of the iliac artery was performed by inflating the balloon at the bifurcation of the abdominal aorta to 3.1 mm diameter and withdrawn to the femoral artery slowly, 3 times (1 min per denudation). Through the main incision, the same balloon was re-inserted into the injured iliac to the bifurcation and inflated to 6-8 atm to occlude proximal blood flow. Immediately after blood flow has been occluded, a IL-10 nanoP3 solution (3×10⁸ total 2 ml) was delivered through the sheath, inflating the artery, and allowed to incubate for 2 minutes before aspirating remaining solution back through the sheath. The sheath was removed, the femoral artery permanently ligated by 3-0 silk suture, followed by removal of the sheath. The exposed area was closed by 3-0 silk suture with individual stiches and double layer sutures.

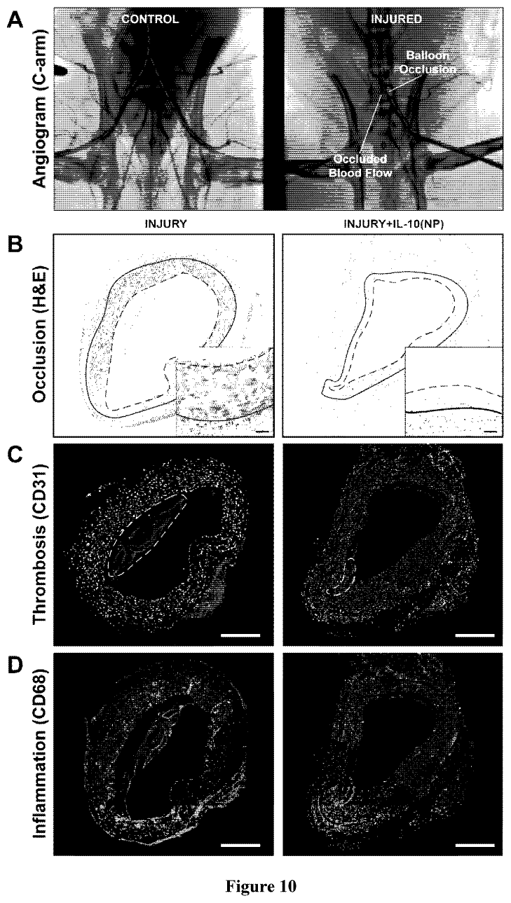

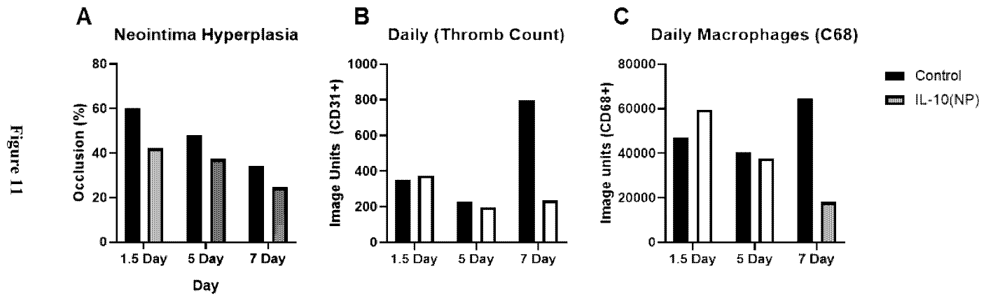

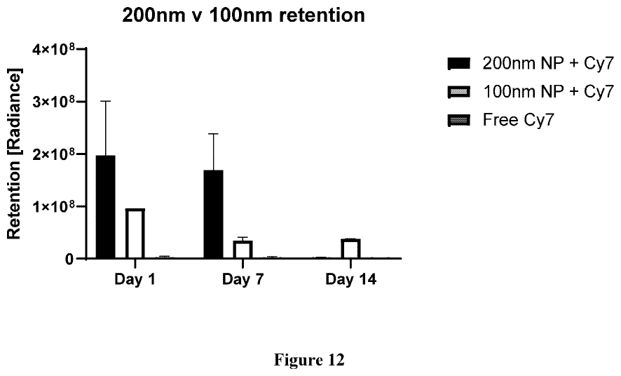

FIG. 10 shows the performance outcomes of IL-10 conjugated 200 nm NP in vivo in a rabbit model of iliac arterial injury. A) rabbit model of iliac arterial injury. B) H&E staining of neointimal hyperplasia. C) CD31 staining of thrombosis (white dotted line). D) CD68 staining of inflammatory macrophage infiltration (white staining). NP3+IL10 treated vessels showed reduced occlusion, thrombosis and inflammation. FIG. 11 shows that NP3+IL-10 treated vessels had reduced hyperplasia over seven days compared to untreated controls. In addition, the incidence of thrombosis increases over seven days and this is significantly reduced in NP3+IL-10 treated vessels. CD68 staining of vessel inflammation shows that NP3+IL-10 reduces the infiltration of pro-inflammatory macrophages and reduces inflammation over seven days compared to non-treated controls.

### Example 7

### Retention of NanoP3 in a Rat Carotid Injury Model

The retention of 200 nm diameter vs 100 nm diameter NanoP3 (NP3) was investigated in a rat carotid injury model.

Cy7 fluorophore was conjugated to either 200 nm diameter of 100 nm diameter NanoP3

The rats were subjected to vessel injury as described in Example 3, except that through the catheter, solutions containing Cy7 bound to 100 nm nanoP3 or Cy7 bound to 200 nm nanoP3 were injected at a concentration of 2×10⁸ nanoP3 in a total volume of roughly 80 μl of RPMI media.

FIG. 12 shows that both 100 nm and 200 nm nanoP3 were retained after seven days, with 100 nm nanoP3 also being retained at two weeks post-delivery.

NanoP3 were detected after 14 days when either size were used. There was some variation on the retention profile depending on the size of the NanoP3 used. Thus, particularly preferred retention times may be achieved by selecting appropriate NanoP3 particle sizes.

1. A method of delivering an agent to a region of a blood vessel in a patient, comprising:
a) conjugating the agent to a nanoparticle to produce a conjugate; and
b) delivering the conjugate to the region of the blood vessel.

2. The method of claim 1, wherein the agent is a biologically active agent or an imaging agent.

3. A method of regulating inflammation or promoting healing in a region of a blood vessel in a patient, comprising:
a) conjugating a biologically active agent to a nanoparticle to produce a conjugate; and
b) delivering the conjugate to the region of the blood vessel.

4. The method according to claim 1, wherein the conjugate is retained in the region of the blood vessel for a period greater than the period unconjugated biologically active agent would be retained in the region of the blood vessel.

5. The method according to claim 4, wherein the conjugate is retained at the site of delivery in the blood vessel for at least 1 day, for at least 5 days, or for at least 14 days.

6. (canceled)

7. (canceled)

8. The method according to claim 1, wherein the biologically active agent is an anti-inflammatory cytokine; an anti-inflammatory drug; a limus drug; a statin drug or an anti-proliferative drug.

9. The method according to claim 8, wherein the biologically active agent is an anti-inflammatory cytokine.

10. The method according to claim 9, wherein the anti-inflammatory cytokine is interleukin-4 or interleukin-10.

11. The method according to claim 8, wherein
the anti-inflammatory drug is sulindac; or wherein
the statin drug is simvastatin; or wherein
the anti-proliferative drug is paclitaxel, Sirolimus or an mTOR inhibitor.

12. The method according to claim 3, wherein the regulation of inflammation is through macrophage polarization.

13. A method of retaining a biologically active agent to a region in a blood vessel in a patient for a period of at least 14 days comprising:
a) conjugating the biologically active agent to a nanoparticle to produce a conjugate; and b) delivering the conjugate to the blood vessel.

14. The method according to claim 1, wherein the conjugate is delivered to the blood vessel using a catheter.

15. The method according claim 14, wherein the catheter is an occlusion perfusion catheter or a sweating balloon catheter.

16. The method according to claim 1, wherein the patient is receiving or has received an endovascular intervention.

17. A conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 5 nm to about 500 nm;
and a biologically active agent selected from the group consisting of: an anti-inflammatory cytokine; anti-inflammatory drug; a statin drug and an anti-proliferative drug.

18. The conjugate according to claim 17, wherein the biologically active agent is an anti-inflammatory cytokine.

19. The conjugate according to claim 18, wherein the anti-inflammatory cytokine is interleukin-4 or interleukin-10.

20. The conjugate according to claim 17, wherein
the anti-inflammatory drug is Sulindac; or wherein
the statin drug is simvastatin; or wherein
the anti-proliferative drug is paclitaxel, Sirolimus or an mTOR inhibitor.

21. A conjugate comprising:
a nanoparticulate polymer with a mean diameter of about 1 nm to about 50 nm and formed from a plasma comprising at least one monomer selected from: an alkene, an alkyne, a cycloalkene, a cycloalkyne, or a mixture thereof; or an aggregate comprising two or more of the nanoparticulate polymers, wherein the aggregate has a mean diameter of about 100 nm to about 200 nm; and
i) interleukin-10; or
ii) Sulindac; or
iii) Sirolimus.

22. A method of treating or preventing vascular injury or vascular disease comprising delivering the conjugate according to claim 17 to a region of a blood vessel in a patient in need thereof.

23. The method according to claim 22, wherein the conjugate is delivered to the region of the blood vessel using a catheter.

24. (canceled)

25. The method according to claim 22, wherein the vascular injury is a result of endovascular intervention.

26. The method according to claim 22, wherein the vascular injury is neointimal hyperplasia or restenosis.

27. The method according to claim 22, wherein the vascular disease is artherosclerosis.

28. (canceled)
